(19) United States
(12) Patent Application Publication
(10) Pub. No.: US 2022/0313623 A1
(43) Pub. Date: Oct. 6, 2022

(54) CANNABINOID FORMULATION AND METHOD OF MAKING THEREOF

(71) Applicant: PET RELEAF IP, LLC, Littleton, CO (US)

(72) Inventor: Joseph Rosentel, Littleton, CO (US)

(21) Appl. No.: 17/703,778

(22) Filed: Mar. 24, 2022

Related U.S. Application Data
(60) Provisional application No. 63/166,093, filed on Mar. 25, 2021.

(51) Int. Cl. A61K 31/05; A61K 31/015; A61K 31/20; A61K 9/48

(52) U.S. Cl. CPC A61K 31/05; A61K 31/015; A61K 31/20; A61K 9/48

(57) ABSTRACT

A formulation for administration to non-human mammals and/or human mammals for the treatment of osteoarthritis includes active ingredients. The active ingredients include (a) an efficacious amount of cannabidiol (“CBD”), a component of a full-spectrum hemp extract (FSHE), or a broad-spectrum hemp extract (BSHE), or a CBD isolate; (b) an efficacious amount of beta-caryophyllene (“BC”); and (c) an efficacious amount caprylic acid (“CA”).

### RELATED APPLICATIONS

This application claims priority to U.S. Provisional Application No. 63/166,093, filed on Mar. 25, 2021, and entitled “CANNABINOID FORMULATION AND METHOD OF MAKING THEREOF,” the entire contents of which are hereby incorporated by reference.

### FIELD

This disclosure relates generally to cannabinoid formulations, and more specifically, to cannabinoid formulations for medical use.

### BACKGROUND

Cannabis is a widely used herb for medication. Medical uses of Cannabis are now legal in many countries. Medical Cannabis can be used for treating and alleviating symptoms associated with a wide variety of symptoms including, but not limited to, pain, glaucoma, arthritis, substance withdrawal, nausea, anxiety, and the like. Other illnesses and symptoms are being treated and alleviated with Cannabis as well. While smoking Cannabis is a prevalent mode of use, other modes of administration may be desirable to reduce effects of smoking on the lungs. For example, U.S. Patent Publication No. 2020/0330378 describes an oral mode of administration in an oil-based form.

### SUMMARY

Broadly, this disclosure relates to new cannabinoid formulations and methods of making the same. The cannabinoid formulations generally can be for medical uses including, but not limited to, administration to non-human mammals and/or human mammals for treatment. The new cannabinoid formulations generally include active ingredients including (a) an efficacious amount of cannabidiol (“CBD”), a component of a full-spectrum hemp extract (FSHE), or a broad-spectrum hemp extract (BSHE), or a CBD isolate; (b) an efficacious amount of beta-caryophyllene (“BC”); and (c) an efficacious amount caprylic acid (“CA”). The CBD may be a component of a full-spectrum hemp extract (FSHE), or a broad-spectrum hemp extract (BSHE), or a CBD isolate. In one embodiment, the new cannabinoid formulations can be in a powder form for inclusion in a capsule for ingestion by a non-human mammal and/or a human mammal. In one embodiment, the new cannabinoid formulations can be administered to canines for medical treatment. The new formulations may facilitate significant increases in quality of life for mammals dealing with osteoarthritis, anxiety, behavioral issues, seizures, anemia, atopy, skin allergies, or the like. For instance, in one embodiment, the new cannabinoid formulation may significantly increase reticulocyte counts in male and/or female mammals (e.g., in canines), as described below. The formulations may also, or alternatively, facilitate increased food consumption. These and other therapeutic effects may be realized due to the administration of the formulations.

### I. Formulations

As noted above, the new cannabinoid formulations generally include active ingredients including (a) an efficacious amount of cannabidiol (“CBD”), a component of a full-spectrum hemp extract (FSHE), or a broad-spectrum hemp extract (BSHE), or a CBD isolate; (b) an efficacious amount of beta-caryophyllene (“BC”); and (c) an efficacious amount caprylic acid (“CA”).

In one embodiment, the active ingredients consist of the CBD, the BC, and the CA.

In one embodiment, a weight ratio of the BC to the CA is from 0.1:1 to 8:1 w/w (BC:CA). In another embodiment, the weight ratio of the BC to the CA is at least 0.2:1, or at least 0.3:1, or at least 0.4:1, or at least 0.5:1, or at least 0.6:1, or at least 0.7:1, or at least 0.8:1, or at least 0.9:1 or at least 1.0:1 w/w (BC:CA). In yet another embodiment, the weight ratio of the BC to the CA is not greater than 7:1, or not greater than 6:1, or not greater than 5:1, or not greater than 4:1, or not greater than 3:1, or not greater than 2.5:1, or not greater than 2:1, or not greater than 1.5:1, or not greater than 1:25 w/w (BC:CA).

In one embodiment, a weight ratio of the CA to the CBD is from 0.1:1 to 8:1 w/w (CA:CBD). In another embodiment, the weight ratio of the CA to the CBD is at least 0.2:1, or at least 0.3:1, or at least 0.4:1, or at least 0.5:1, or at least 0.6:1, or at least 0.7:1, or at least 0.8:1, or at least 0.9:1 or at least 1.0:1 w/w (CA:CBD). In yet another embodiment, the weight ratio of the CA to the CBD is not greater than 7:1, or not greater than 6:1, or not greater than 5:1, or not greater than 4:1, or not greater than 3:1, or not greater than 2.5:1, or not greater than 2:1, or not greater than 1.5:1, or not greater than 1:25 w/w (CA:CBD).

In one embodiment, the formulation includes at least one of a bitterness masker; an antioxidant; and an excipient. In another embodiment, the bitterness masker includes mushroom extract. In yet another embodiment, the antioxidant includes at least one of rosemary extract, green tea, acerola, a tocopherol, a free radical quencher, and a chelator. In another embodiment, the excipient is vegetable-based. In another embodiment, the excipient is corn-based.

In one embodiment, the formulation is a powder consisting of the active ingredients, and one of the bitterness masker, the antioxidant, and the excipient. In another embodiment, the formulation is a powder consisting of the active ingredients, and two of the bitterness masker, the antioxidant, and the excipient. In yet another embodiment, the formulation is a powder consisting of the active ingredients, and all of the bitterness masker, the antioxidant, and the excipient.

In one embodiment, a capsule includes the formulation and from 1 to 200 mg of the CBD; from 1 to 200 mg of the BC; and from 1 to 200 mg of the CA.

In one embodiment, the capsule is a vegan capsule. In another embodiment, the capsule is a non-vegan capsule.

In one embodiment, the capsule includes 10 mg of the CBD; 10 mg of the BC; and 10 mg of the CA. In another embodiment, the capsule includes 30 mg of the CBD; 30 mg of the BC; and 30 mg of the CA.

In one embodiment, the capsule has a total capsule weight from 200 mg to 600 mg. In another embodiment, the total capsule weight is from 250 mg to 550 mg. In yet another embodiment, the total capsule weight is from 300 mg to 515 mg.

In one embodiment, the capsule has a total fill weight, wherein the total fill weight is from 150 mg to 500 mg. In one embodiment, the total fill weight is from 200 mg to 450 mg. In another embodiment, the total fill weight is from 240 mg to 420 mg.

### II. Methods of Making

The new cannabinoid formulations may be processed in a liquid form to create a liquid cannabinoid formulation. In one embodiment, the liquid formulation may be mixed with a powder to create a powder cannabinoid formulation.

In one embodiment, and referring now to FIG. 1, a method 100 of making the formulation includes 105 producing a liquid-based mixture. The liquid based mixture includes (i) full-spectrum hemp extract oil; (ii) liquid beta-caryophyllene; and (iii) liquid caprylic acid.

In one embodiment, the method 100 may optionally include 110 adding a liquid antioxidant to the liquid-based mixture. In another embodiment, the method 100 may optionally include 115 adding a liquid bitterness masking agent to the liquid-based mixture. In yet another embodiment, the method 100 may include 120 adding silicon dioxide to the liquid-based mixture.

In one embodiment, the method 100 may optionally include 125 adding a powder-based excipient to the liquid-based mixture. The powder-based excipient absorbs the liquid-based mixture, thereby producing the formulation comprising the CBD, the BC, and the CA into a powder for capsule filling.

### III. Methods of Treatment

In one embodiment, a method of treatment for osteoarthritis in a mammal includes administration of a therapeutically efficacious amount of the formulation.

In one embodiment, a method of treatment for anxiety in a mammal includes administration of a therapeutically efficacious amount of the formulation.

In one embodiment, a method for treatment for seizures in a mammal includes administration of a therapeutically efficacious amount of the formulation.

In one embodiment, method of treatment for anemia in a mammal includes administration of a therapeutically efficacious amount of the formulation.

In one embodiment, a method of treatment for atopy or allergic skin disease in a mammal includes administration of a therapeutically efficacious amount of the formulation.

In one approach, the administration of the therapeutically efficacious amount of the formulation includes a capsule including the formulation and from 1 to 200 mg of the CBD; from 1 to 200 mg of the BC; and from 1 to 200 mg of the CA.

In one embodiment, the above methods of treatment are for a human mammal. In another embodiment, the above methods of treatment are for a non-human mammal. In yet another embodiment, the above methods of treatment are for a canine.

### IV. Properties

As noted above, the new cannabinoid formulations can be used in methods of treatment of human and/or non-human mammals.

In one embodiment, a detectable amount of CBD is present in a mammal in less than 30 minutes following administration.

In one embodiment, a first mean concentration of the CBD in plasma of the mammal tested using liquid chromatography-mass spectrometry 30 minutes following administration on Day 1 is 5 to 30 ng/mL. In one embodiment, a second mean concentration of the CBD remaining in the plasma of the mammal tested using liquid chromatography-mass spectrometry 12 hours after administration on Day 1 varies by less than 40% of the first mean concentration.

In one embodiment, the therapeutically efficacious amount of the CBD is 10 mg. In another embodiment, the therapeutically efficacious amount of the CBD is 30 mg.

In one embodiment, a therapeutic amount of CBD is present in the mammal tested using liquid chromatography-mass spectrometry 30 to 60 minutes following administration on Day 1.

In one embodiment, a reticulocyte count on Day 29 after administration on Day 1 is 1.7 to 1.9 times a baseline reticulocyte count for a male mammal using a hematology test. In one embodiment, a reticulocyte count on Day 29 after administration on Day 1 is 1.2 to 1.5 times a baseline reticulocyte count for a female mammal using a hematology test.

In one embodiment, a food consumption measured in g/mammal/day for a male mammal increases by 5 to 30% relative to a baseline food consumption over 28 days. In one embodiment, a food consumption measured in g/mammal/day for a female mammal increases by 25 to 90% relative to a baseline food consumption over 28 days.

### V. Definitions

As used herein, a “total capsule weight” includes a total weight of all ingredients along with a weight of the capsule.

As used herein, a “total fill weight” includes a total weight of all ingredients without inclusion of the weight of the capsule.

As used herein, a “baseline consumption” includes a food consumption rate in g/mammal/day for a period immediately preceding consumption of the formulation described herein. In one embodiment, the baseline consumption can be based on a food consumption rate for 1 day immediately preceding consumption of the formulation described herein. In one embodiment, the baseline consumption can be based on a food consumption rate for 2 days immediately preceding consumption of the formulation described herein.

As used herein, a “baseline reticulocyte count” includes a reticulocyte count determined from a blood sample of a mammal using a hematology test 2 days prior to administration of the new cannabinoid formulations described herein.

As used herein, a “hematology test” includes testing a blood sample using an Advia® 120 Hematology System, commercially available from Siemens Healthcare GmbH.

### VI. Miscellaneous

These and other aspects, advantages, and novel features of this new technology are set forth in part in the description that follows and will become apparent to those skilled in the art upon examination of the following description and figures or may be learned by practicing one or more embodiments of the technology provided for by the present disclosure.

The figures constitute a part of this specification and include illustrative embodiments of the present disclosure and illustrate various objects and features thereof. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present embodiments.

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

Throughout the specification and claims, the following terms take the meanings explicitly associated herein, unless the context clearly dictates otherwise. The phrases “in one embodiment” and “in one embodiment” as used herein do not necessarily refer to the same embodiment(s), though they may. Furthermore, the phrases “in another embodiment” and “in some other embodiments” as used herein do not necessarily refer to a different embodiment, although they may. Thus, various embodiments of the disclosure may be readily combined, without departing from the scope or spirit of the disclosure.

In addition, as used herein, the term “or” is an inclusive “or” operator and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references, unless the context clearly dictates otherwise. The meaning of “in” includes “in” and “on”, unless the context clearly dictates otherwise.

While a number of embodiments of the present disclosure have been described, it is understood that these embodiments are illustrative only, and not restrictive, and that many modifications may become apparent to those of ordinary skill in the art. Further still, unless the context clearly requires otherwise, the various steps may be carried out in any desired order, and any applicable steps may be added and/or eliminated.

### BRIEF DESCRIPTION OF THE DRAWINGS

Reference is made to the drawings that form a part of this disclosure, and which illustrate the embodiments in which the devices and methods described herein can be practiced.

FIG. 1 is a flowchart of a method for making a cannabinoid formulation, according to one embodiment.

FIG. 2 is a plot showing mean concentration (ng/mL) of CBD in plasma of canines following oral administration at day 1, according to one embodiment.

FIG. 3 is a plot showing mean concentration (ng/mL) of CBD in plasma of canines following oral administration at day 28, according to one embodiment.

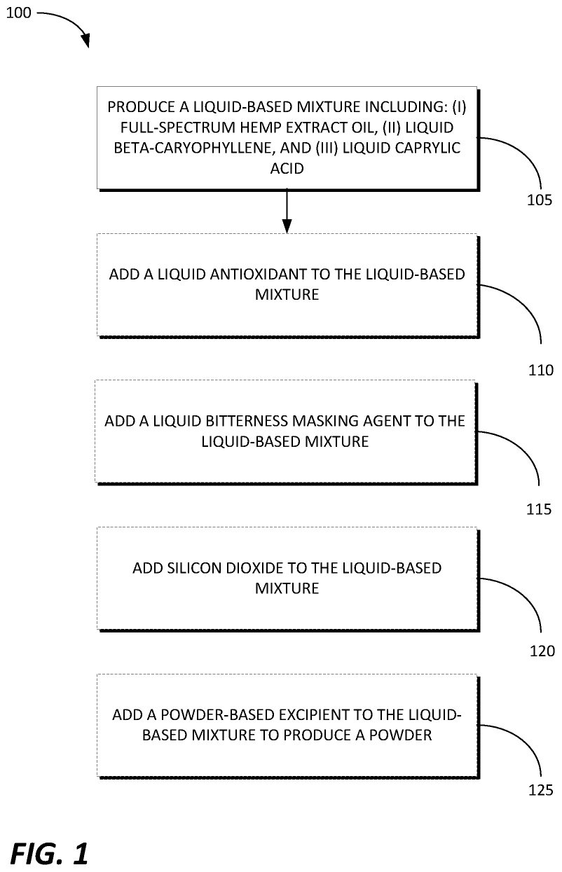

### DETAILED DESCRIPTION

### Example 1

A full-spectrum hemp extract cannabinoid oil is mixed with caprylic acid, beta-caryophyllene, and a liquid antioxidant to prepare a liquid mixture. In one embodiment, the liquid antioxidant can include, but is not limited to, DURALOX®, commercially available from Kalsec Inc. of Kalamazoo, Mich. A liquid bitterness masking agent is then added to the liquid mixture. In one embodiment, the liquid bitterness masking agent can include, but is not limited to, CLEARTASTE, commercially available from MycoTechnology, Inc. of Aurora, Colo. Next, a vegetable-based excipient and silicon dioxide are added until the mixture turns into a powder. In one embodiment, the vegetable-based excipient includes, but is not limited to, FIBERSOL®, commercially available from Matsutani Chemical Industry Co, Ltd. of Japan. In one embodiment, the silicon dioxide includes, but is not limited to, FujiSil™, commercially available from Fuji Chemical Industries USA, Inc.

The powder is then placed into capsules. The amounts of the active ingredients within the capsules are shown in Table 1, below.

TABLE 1
Capsule Active Ingredients

| Material | Small Capsule | | Large Capsule | |
|---|---|---|---|---|
| Full Spectrum hemp extract | 13.6 | mg | 40.8 | mg |
| Cannabinoid (CBD) content via | 10 | mg | 30 | mg |
| full-spectrum hemp extract | | | | |
| Beta-caryophyllene | 10 | mg | 30 | mg |
| Caprylic acid | 10 | mg | 30 | mg |

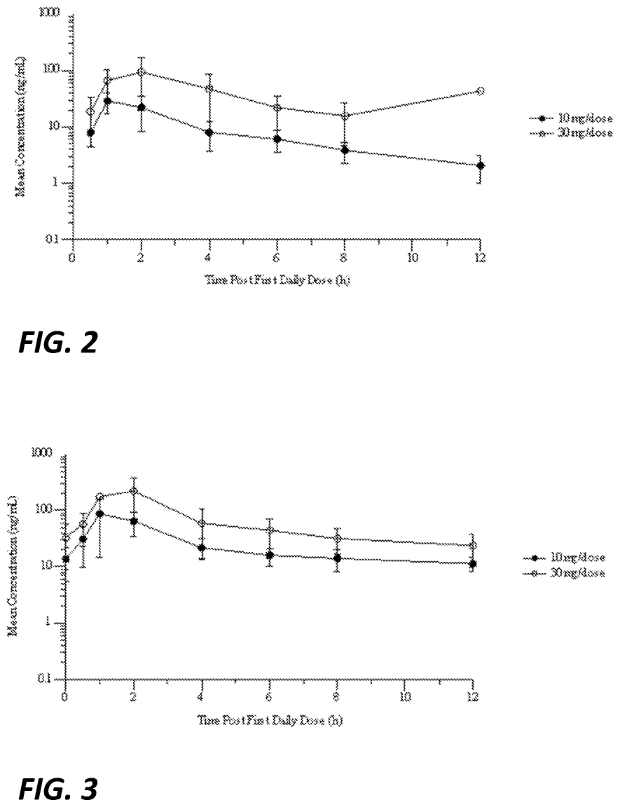

A pharmacokinetic and toxicokinetic study was conducted to assess the exposure to CBD through blood samples following the first oral administration (Day 1) of either 1 capsule (10 mg CBD) or 3 capsules (30 mg CBD) twice daily (approximately 12 hours apart) and on the last morning of oral administration (Day 28) to male and female Beagle dogs, weighing 5-15 kgs, for 28 days. The blood samples were evaluated for mean concentration (ng/mL) of the CBD using liquid chromatography-mass spectrometry. The capsules administered to the canines resulted in fast absorption with detectable levels of CBD in the plasma in less than 30 minutes following administration of the first daily dose (Day 1) and therapeutic levels in less than 60 minutes following administration of the first daily dose (Day 1). Therapeutic levels of CBD were present in the plasma for the entire 12 hour dose interval (Day 1). FIGS. 2 and 3 show the mean concentration (ng/mL) of CBD in plasma of the canines following oral administration at Day 1 (FIG. 2) and at Day 28 (FIG. 3). The male and female dogs were combined, showing a single line for each concentration.

A safety study was conducted in male and female Beagle dogs, weighing 5-15 kgs, by dosing orally at 1× the recommendation twice daily for 28 days (1 capsule=10 mg CBD, 10 mg beta-caryophyllene, and 10 mg caprylic acid; 20 mg CBD/day) and 3× the recommendation twice daily for 28 days (3 capsules=30 mg CBD, 30 mg beta-caryophyllene, and 30 mg caprylic acid; 60 mg CBD/day). The capsules administered to the canines resulted in no clinical observations, no body weight changes (Table 2), higher food consumption (Table 3), and increased reticulocyte counts (Table 4).

Table 2 shows results of the weight gain changes for male and female canines in the safety study.

TABLE 2
Body Weight Gain

| Body | | |
|---|---|---|
| Weight | CBD Dose Level | |
| Gain | Male | Female |

| (kg) | 20 mg/day | 60 mg/day | 20 mg/day | 60 mg/day |
|---|---|---|---|---|
| Day −7-1 | −0.27 | −0.33 | −0.17 | −0.47 |
| Day 1-28 | 0.03 | 0.23 | 0.00 | 0.27 |

Table 3 shows results of the food consumption for male and female canines in the safety study.

TABLE 3
Food Consumption

| Food | CBD Dose Level | | | |
|---|---|---|---|---|
| Consumption | Male | | Female | |
| (g/animal/day) | 20 mg/day | 60 mg/day | 20 mg/day | 60 mg/day |
| Day −2-1 | 204.67 | 189.67 | 145.56 | 104.78 |
| Day 1-28 | 221.25 (8%) | 241.07(27%) | 189.05(30%) | 195.02(86%) |

Table 4 shows results of the reticulocyte counts for male and female canines in the safety study. The reticulocyte values in Table 4 were determined using an Advia® 120 Hematology System commercially available from Siemens Healthcare GmbH.

TABLE 4
Hematology Parameter Changes

| Parameter | CBD Dose Level | | | |
|---|---|---|---|---|
| RETIC | Males | | Females | |
| (10\hat{ }9/L) | 20 mg/day | 60 mg/day | 20 mg/day | 60 mg/day |
| Day −2 | 37.13 | 42.73 | 32.53 | 46.13 |
| Day 15 | 39.87 | 38.07 | 39.87 | 38.07 |
| Day 29 | 65.33 (1.76X) | 75.77 (1.77X) | 45.77(1.41X) | 57.37 (1.24X) |

All other hematological parameters were normal for both the 1× and 3× groups.

The serum chemistry parameters were normal for all parameters, except alkaline phosphatase (ALP) which was mildly elevated in the 3× dosage group.

Table 5 shows the ALP results for male and female canines in the safety study. The ALP values in Table 5 were determined using an Advia® 1800 Chemistry System commercially available from Siemens Healthcare GmbH.

TABLE 5
Serum Chemistry Parameter Changes

| | CBD Dose Level | | | |
|---|---|---|---|---|
| Parameter | Males | | Females | |
| ALP (U/L) | 20 mg/day | 60 mg/day | 20 mg/day | 60 mg/day |
| Day −2 | 44.7 | 77.3 | 43.7 | 51.3 |
| Day 15 | 70.0 (1.57X) | 113.3 (1.47X) | 58.7 (1.34X) | 81.7 (1.59X) |
| Day 29 | 67.3 (1.51X) | 106.7 (1.38X) | 64.3 (1.47X) | 119.3 (2.33) |

All urinalysis parameters were normal for both the 1× dosage and 3× dosage groups.

The capsules can be administered to canines twice daily to treat pain and inflammation due to osteoarthritis. Dogs treated with this formulation have a significant decrease in pain score, have significant reduction in inflammatory biomarkers, and have a significant increase in quality of life compared to a placebo group.

The capsules can be administered to canines twice daily to treat anxiety and behavior issues. Dogs treated with this formulation have a significant decrease in anxiety, have significant reduction in unwanted behavior issues, and have a significant increase in quality of life compared to a placebo group.

The capsules can be administered to canines twice daily to treat seizure disorders. Dogs treated with this formulation have a significant decrease in the number of seizures, duration of seizures, severity of seizures, and have significant increase in quality of life compared to a placebo group.

The capsules can be administered to canines twice daily to treat anemia. Dogs treated with this formulation have an increase in reticulocyte counts, maintain hematocrit levels, and have a significant increase in quality of life compared to a placebo group.

The capsules can be administered to canines twice daily to treat atopy or allergic skin disease. Dogs treated with this formulation have a significant decrease in itching, have significant reduction in hot spots, have a significant reduction in alopecia, and have a significant increase in quality of life compared to a placebo group.

It is to be appreciated that the above capsules can be administered to non-human mammals (e.g., canines as above) or to human-mammals.

While various embodiments of the present disclosure have been described in detail, it is apparent that modifications and adaptations of those embodiments will occur to those skilled in the art. However, it is to be expressly understood that such modifications and adaptations are within the spirit and scope of the present disclosure.

What is claimed is:

1. A formulation for administration to mammals, the formulation comprising active ingredients, wherein the active ingredients comprise:
(a) an efficacious amount of cannabidiol (“CBD”), a component of a full-spectrum hemp extract (FSHE), or a broad-spectrum hemp extract (BSHE), or a CBD isolate;
(b) an efficacious amount of beta-caryophyllene (“BC”); and
(c) an efficacious amount caprylic acid (“CA”).

2. The formulation of claim 1, wherein the active ingredients consist of the CBD, the BC, and the CA.

3. The formulation of claim 1, wherein a weight ratio of the BC to the CA is from 0.1:1 to 8:1 w/w (BC:CA).

4. The formulation of claim 1, wherein a weight ratio of the CA to the CBD is from 0.1:1 to 8:1 w/w (CA:CBD).

5. The formulation of claim 1, comprising at least one of:
a bitterness masker;
an antioxidant; and
an excipient.

6. The formulation of claim 5, wherein at least one of:
the bitterness masker comprises mushroom extract;
the antioxidant comprises at least one of: rosemary extract, green tea, acerola, a tocopherol, a free radical quencher, and a chelator;
the excipient is vegetable-based; or
the excipient is corn-based.

7. The formulation of claim 1, wherein the mammals are canines.

8. A capsule for administration to mammals, the capsule comprising:
(a) an efficacious amount of cannabidiol (“CBD”), a component of a full-spectrum hemp extract (FSHE), or a broad-spectrum hemp extract (BSHE), or a CBD isolate;
(b) an efficacious amount of beta-caryophyllene (“BC”); and
(c) an efficacious amount caprylic acid (“CA”);
wherein the capsule comprises:
from 1 to 200 mg of the CBD;
from 1 to 200 mg of the BC; and
from 1 to 200 mg of the CA.

9. The capsule of claim 8, wherein the capsule comprises:
10 mg of the CBD;
10 mg of the BC; and
10 mg of the CA.

10. The capsule of claim 8, wherein the capsule comprises:
30 mg of the CBD;
30 mg of the BC; and
30 mg of the CA.

11. A method comprising:
(a) producing a liquid-based mixture comprising:
(i) full-spectrum hemp extract oil;
(ii) liquid beta-caryophyllene;
(iii) liquid caprylic acid; and
(b) adding a powder-based excipient to the liquid-based mixture, wherein the powder-based excipient absorbs the liquid-based mixture, thereby creating a powder.

12. The method of claim 11, comprising adding a liquid antioxidant to the liquid-based mixture.

13. The method of claim 11, comprising adding a liquid bitterness masking agent to the liquid-based mixture.

14. The method of claim 11, comprising adding silicon dioxide to the liquid-based mixture.
